(19) United States
(12) Patent Application Publication
(10) Pub. No.: US 2007/0167689 A1
(43) Pub. Date: Jul. 19, 2007

(54) METHOD AND SYSTEM FOR ENHANCING A USER EXPERIENCE USING A USER'S PHYSIOLOGICAL STATE

(75) Inventors: Padmaja Ramadas, Davie, FL (US); Ronald J. Kelley, New Brunswick, NJ (US); Sivakumar Muthuswamy, Tower Lakes, IL (US); Robert W. Pennisi, Boca Raton, FL (US); Steven D. Pratt, Yardley, PA (US)

Correspondence Address: MOTOROLA, INC;INTELLECTUAL PROPERTY SECTION, LAW DEPT, 8000 WEST SUNRISE BLVD, FT LAUDERDAL, FL, 33322, US

(73) Assignee: Motorola, Inc., Schaumburg, IL (US)

(21) Appl. No.: 11/615,951

(22) Filed: Dec. 23, 2006

Related U.S. Application Data
(62) Division of application No. 11/097,711, filed on Apr. 1, 2005.

(51) Int. Cl. A61B 5/00; A61B 5/02; A61B 5/08; A61B 5/103

(52) U.S. Cl. 600/300; 128/905; 600/500; 600/549; 600/595; 600/529; 600/306

(57) ABSTRACT

A method (50) of altering content provided to a user includes the steps of creating (60) a user profile based on past physiological measurements of the user, monitoring (74) at least one current physiological measurement of the user, and altering (82) the content provided to the user based on the user profile and the at least one current physiological measurement. The user profile can be created by recording a plurality of inferred or estimated emotional states (64) of the user which can include a time sequence of emotional states, stimulus contexts for such states, and a temporal relationship between the emotional state and the stimulus context. The content can be altered in response to the user profile and measured physiological state by altering at least one among an audio volume, a video sequence, a sound effect, a video effect, a difficulty level, a sequence of media presentation.

### RELATED APPLICATION

This application is a Divisional of application Ser. No. 11/097,711, filed Apr. 1, 2005. Applicant claims priority thereof.

### FIELD OF THE INVENTION

This invention relates generally to providing content to a user, and more particularly to altering content based on a user's physiological condition or state.

### BACKGROUND OF THE INVENTION

Medical, gaming, and other entertainment devices discussed in various U.S. Patents and publications discuss measuring a user's physiological state in an attempt to manipulate an application running in the respective devices. Each existing system attempts to determine an emotional state based on real-time feedback. Existing parameters such as pulse rate or skin resistivity or skin conductivity (among others) may not always be the best and most accurate predictors of an emotional state of a user.

### SUMMARY OF THE INVENTION

Embodiments in accordance with the present invention can provide a user profile along with physiological data for a user to enhance a user experience on an electronic device such as a gaming device, a communication device, medical device or practically any other entertainment device such as a DVD player.

Embodiments can include a software method of altering a sequence of events triggered by physiological state variables along with user profiles, and an apparatus incorporating the software and sensors for monitoring the physiological characteristics of the user. Such embodiments can combine sensors for bio-monitoring, electronic communication and/or multi-media playback devices and computer algorithm processing to provide an enhanced user experience across a wide variety of products.

In a first embodiment of the present invention, a method of altering content provided to a user includes the steps of creating a user profile based on past physiological measurements of the user, monitoring at least one current physiological measurement of the user, and altering the content provided to the user based on the user profile and the at least one current physiological measurement. The user profile can be created by recording a plurality of inferred or estimated emotional states of the user which can include a time sequence of emotional states, stimulus contexts for such states, and a temporal relationship between the emotional state and the stimulus context. Stimulus context can include one or more among lighting conditions, sound levels, humidity, weather, temperature, other ambient conditions, and/or location. The user profile can further include at least one among user id, age, gender, education, temperament, and past history with the same or similar stimulus class. The step of monitoring can include monitoring at least one among heart rate, pulse, blood oxygen levels, temperature, eye movements, body movements, breathing rates, audible vocalizations, skin conductivity, skin resistivity, Galvanic skin responses, audio level sensing, or force sensing. The content can be altered in response to the user profile and measured physiological state by altering at least one among an audio volume, a video sequence, a sound effect, a video effect, a difficulty level, a sequence of media presentation.

In a second embodiment of the present invention, another method of altering content provided to a user can include the steps of retrieving a user profile based on past physiological measurements of the user, monitoring at least one current physiological measurement of the user, and altering the content provided to the user based on the user profile and the at least one current physiological measurement. The user profile can include at least one among a user preference, a user id, age, gender, education, temperament, and a past history with the same or similar stimulus class. The user profile can further include recordings of at least one or more among a plurality of inferred or estimated emotional states of the user, a time sequence of emotional states, stimulus contexts, and a temporal relationship between the emotional state and the stimulus context. The user profile can also include recorded environmental conditions among lighting conditions, sound levels, humidity, weather, temperature, and location. Among the physiological conditions monitored can include heart rate, pulse, blood oxygen levels, temperature, eye movements, body movements, breathing rates, audible vocalizations, skin conductivity, skin resistivity, Galvanic skin responses, audio level sensing, or force sensing.

In a third embodiment of the present invention, an electronic device can include a sensor for monitoring at least one current physiological measurement of a user, a memory for storing a user profile containing information based on past physiological measurements of the user, a presentation device for providing a presentation to the user, and a processor coupled to the sensor and the presentation device. The processor can be programmed to alter the presentation based on the user profile and the at least one current physiological measurement of the user. As discussed with reference to other embodiments, the user profile can include at least one or more among a plurality of inferred or estimated emotional states of the user, a time sequence of emotional states, stimulus contexts, and a temporal relationship between the emotional state and the stimulus context. The user profile can further include recorded environmental conditions selected among the group of lighting conditions, sound levels, humidity, weather, temperature, or location. The user profile can also include at least one among a user id, age, gender, education, temperament, and past history with the same or similar stimulus class. The sensor(s) for monitoring can include at least one sensor for monitoring among heart rate, pulse, blood oxygen levels, temperature, eye movements, body movements, breathing rates, audible vocalizations, skin conductivity, skin resistivity, Galvanic skin responses, audio level sensing, location, or force sensing. The electronic device can further include a receiver and a transmitter coupled to the processor and the presentation device comprises at least one among a display, an audio speaker, a vibrator, or other sensory output device. The electronic device can be a mobile phone, a smart phone, a PDA, a laptop computer, a desktop computer, an electronic gaming device, a gaming controller, a remote controller, a DVD player, an MP3 player, a CD player or any other electronic device that can enhance a user's experience using the systems and techniques disclosed herein.

Other embodiments, when configured in accordance with the inventive arrangements disclosed herein, can include a system for performing and a machine readable storage for causing a machine to perform the various processes and methods disclosed herein.

### BRIEF DESCRIPTION OF THE DRAWINGS

FIG. 1 is a block diagram of an electronic device using a user's physiological state in accordance with an embodiment of the present invention.

FIG. 2 is a flow chart illustrating a method of using a user profile and a user's physiological state to alter content in accordance with an embodiment of the present invention.

### DETAILED DESCRIPTION OF THE DRAWINGS

While the specification concludes with claims defining the features of embodiments of the invention that are regarded as novel, it is believed that the invention will be better understood from a consideration of the following description in conjunction with the figures, in which like reference numerals are carried forward.

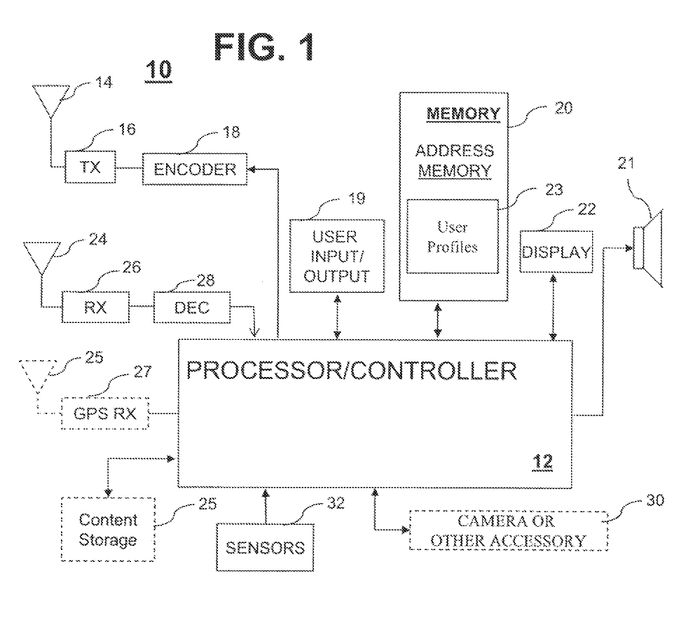

Referring to FIG. 1, a communication device 10 such a mobile telephone or camera phone (or any other electronic device having a user interface) can include a processor 12 programmed to function in accordance with the described embodiments of the present invention. The communication device 10 can essentially be any media playback device or input device having sensors. Examples of typical electronic communication and/or multi-media playback devices within contemplation of the various embodiments herein can include, but is not limited to cell-phones, smart-phones, PDAs, home computers, laptop computers, pocket PCs, DVD players, personal audio/video playback devices such as CD & MP3 players, remote controllers, and electronic gaming devices and accessories.

The portable communication device 10 can optionally include (particularly in the case of a cell phone or other wireless device) an encoder 18, transmitter 16 and antenna 14 for encoding and transmitting information as well as an antenna 24, receiver 26 and decoder 28 for receiving and decoding information sent to the portable communication device 10. The communication device 10 can further include a memory 20, a display 22 for displaying a graphical user interface or other presentation data, and a speaker 21 for providing an audio output. The memory 20 can further include one or more user profiles 23 for one or more users to enhance the particular user's experience as will be further explained below. Additional memory or storage 25 (such as flash memory or a hard drive) can be included to provide easy access to media presentations such as audio, images, video or multimedia presentations for example. The processor or controller 12 can be further coupled to the display 22, the speaker 21, the encoder 18, the decoder 28, and the memory 20. The memory 20 can include address memory, message memory, and memory for database information which can include the user profiles 23.

Additionally, the communication device 10 can include user input/output device(s) 19 coupled to the processor 12. The input/output device 19 can be a microphone for receiving voice instructions that can be transcribed to text using voice-to-text logic for example. Of course, input/output device 19 can also be a keyboard, a keypad, a handwriting recognition tablet, or some other Graphical User Interface for entering text or other data. If the communication device is a gaming console, the input/output device 19 could include not only the buttons used for input, but a vibrator to provide haptics for a user in accordance with an embodiment herein. Optionally, the communication device 10 can further include a GPS receiver 27 and antenna 25 coupled to the processor 12 to enable location determination of the communication device. Of course, location or estimated location information can be determined with just the receiver 26 using triangulation techniques or identifiers transmitted over the air. Further note, the communication device can include any number of applications and/or accessories 30 such as a camera. In this regard, the camera 30 (or other accessory) can operate as a light sensor or other corresponding sensor. The communication device 10 can include any number of specific sensors 32 that can include, but is not limited to heart rate sensors (i.e. ecg, pulse oximetry), blood oxygen level sensors (i.e. pulse oximetry), temperature sensors (i.e. thermocouple, IR non contact), eye movement and/or pupil dilation sensors, motion sensing (i.e. strain gauges, accelerometers, rotational rate meters), breathing rate sensors (i.e. resistance measurements, strain gauges), Galvanic skin response sensors, audio level sensing (i.e. microphone), force sensing (i.e. pressure sensors, load cells, strain gauges, piezoelectric). Each of these sensors can measure a physiological state or condition or the user and/or an environmental condition that will assist the communication device 10 to infer an emotional state of the user.

Many different electronic products can enhance a user's experience with additional interactions through biometric sensors or other sensors. Most current products fail to provide a means for a device to detect or react to a user's physiological state. In gaming and electronic entertainment applications for example, knowing the physiological state of the user and altering the game or entertainment accordingly should generally lead to greater customer satisfaction. For example, characteristics of a game such as difficulty level, artificial intelligence routines, and/or a sequence of events can be tailored to an individual response of the user in accordance to the game's events. Electronic entertainment software such as videogames, DVD movies, digital music and sound effects could be driven by the user's physiological reaction to the media. For example, the intensity of a DVD horror movie could evolve during playback based upon the user's response to frightening moments in the film. Computer software or multi-media content can branch to subroutines or sub-chapters based on physiological sensor inputs. The user can further customize preferences, tailoring the amount of fright, excitement, suspense, or other desired (or undesired) emotional effect, based on specific physiological sensor inputs. A profile can be maintained and used with current physiological measurements to enhance the user experience. For example, user interface software and/or artificial intelligence routines can be used to anticipate a user action based on stored historical actions taken under similar physiological conditions that can be stored in a profile. In this manner, the device learns from historical usage patterns. Thus, embodiments herein can alter at least one among an audio volume, a video sequence, a sound effect, a video effect, a difficulty level, a sequence of media presentation (as examples) in response to the user profile and at least one current physiological measurement.

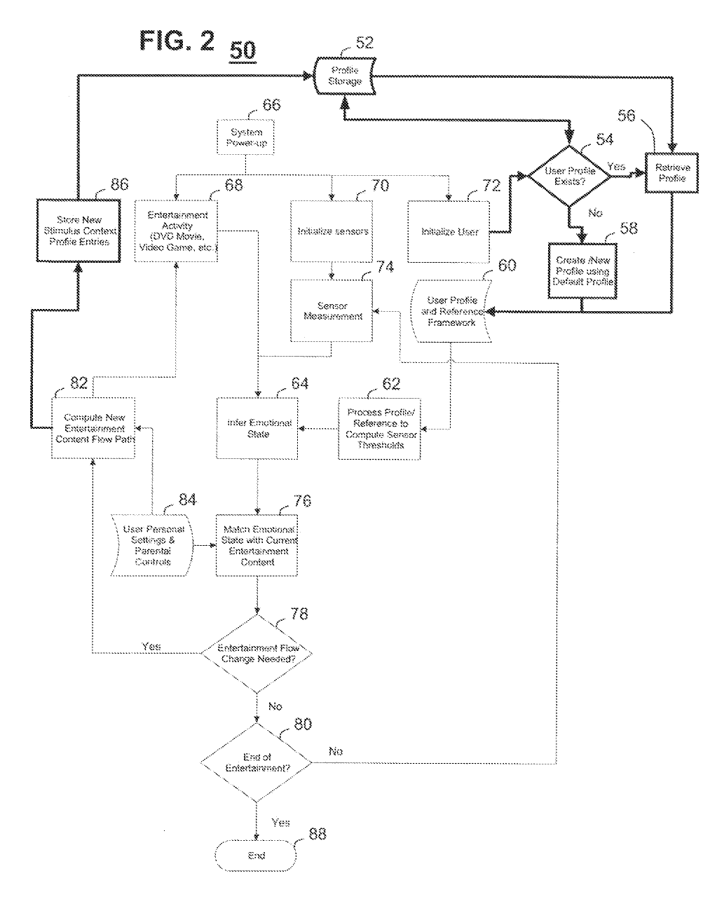

During a typical entertainment experience, the effect of the experience can be optimized by matching entertainment content and flow of the content in response to the observed emotional state of the audience or particular user. As stated above, the emotional state can be derived from physiological measurements such as heart rate, pulse, eye or pupil movements, body movements, and other sensed data. Referring to FIG. 2, an algorithm 50 starts at the power-up cycle of the entertainment device at step 66 and initializes the entertainment activity at step 68, the physiological sensors at step 70 and optionally also identifies the user at step 72, and also identifies the environment, location, time and or other factors related to context using a sensor measurement at step 74. The algorithm can utilize a mathematical model (neural networks, state machines, simple mathematical model, etc.), which measure particular physiological responses of the user to compute a metric which will be defined as an emotion or pseudo emotion at step 64. The defined emotion may or may not correlate to what is commonly accepted by experts in the study of emotion as an actual emotion. It would be desirable if there were a strong correlation for clinical applications, but for the purposes of a game, a pseudo emotion would be sufficient. In addition the algorithm will correlate the emotion or pseudo emotion with performance in a task such as a game or it could simply provide feedback to the user for a game at step 74. If the emotional state indicates a change, then at decision block 78, the algorithm 50 can request a change in the entertainment flow or content to better suit the emotional or perceived emotional state. If no change is required in the entertainment flow at decision block 78, then at decision block 80 the algorithm ends at step 88 if the entertainment program is complete or the algorithm continues to sense the physiological and/or environmental state or conditions at step 74. Using the emotional state and any personal user settings or parental controls from step 84, a new entertainment flow path can be computed at step 82. The computed flow and any new stimulus context can be provided to update the entertainment activity at step 68. The new stimulus context can also be used to update a profile at step 86 which is stored in a profile storage at step 52. Note, the emotion or pseudo emotion can be used to enhance the user interface such as provide pleasing colors or fonts without direct interaction with the user. For example, the user may find that a “times roman” font may “feel” better in the day time or a “courier” font may “feel” better in the evening even though the user may not be consciously aware of such feelings. The device therefore is capable of identifying the emotional response to any changes in the device, game, or user interface.

The user identification can be based on a login process or through other biometric mechanisms. The user creates a profile or the device could create a user profile automatically. In this regard, at decision block 54, if a user profile exists, then it is retrieved at step 56 from the profile storage 52. If no user profile exists at decision block 54, then a new profile using a default profile can be created at step 58. The profile can generally be a record of various inferred/estimated emotional states of the user (combination of one or more emotional states), time sequence of the emotional states and various stimulus contexts (such as scene in a movie, state of a video game, type of music played, difficulty of a programming task, etc.) and the temporal relationship between the inferred state and the stimulus context. This profile can also include such external environmental information such as ambient conditions (lighting, loudness, humidity, weather, temperature, location, GPS (possibly indicate a particular location where the user becomes excited) or other inputs). In addition, the profile can include user identification information or a reference framework at step 60 that can include among user ID, age, gender, education, temperament, past history with the same or similar stimulus class or other pertinent framework data for the user. The user profile is stored and can be saved in a variety of forms from simple object attribute data to more sophisticated probability density functions associated with neural networks or genetic algorithms. The complexity and sophistication of the storage method is based on the device resource context and added value of the premium features. In one embodiment, the profile can be stored as a probability based profile mechanism that can suitably configure to new stimulus contexts and unpredictable inferred emotional states.

The algorithm 50 can start with a default profile that evolves in sophistication over time for a particular user or user class. The profile can be hierarchical in nature with single or multiple inheritances. For example, the profile characteristics of gender will be inherited by all class members and each member of the class will have additional profile characteristics that are unique to the individual that evolves over time.

Based on the user identification and other profile data, the sensor thresholds corresponding to a particular emotional state are set at step 62. As the entertainment progresses, the physiological sensors are monitored at step 74 and the emotional state of the user is inferred at step 64 using the measured values. The inferred emotional state is matched to the type of entertainment content at step 76 and a decision is made about the need to change content flow at decision block 78 as described above. The decision can be based on tracking emotional state over a period of time (using the profiles and the instantaneous values) as opposed to the instantaneous values alone. The decision at decision block 78 can also be influenced by any user settings or parental controls in effect in the entertainment system at step 84. Note, a measured response of the user can be represented by an emoticon (i.e., icons or characters representing smiley, grumpy, angry, or other faces a commonly used in instant messaging. Also, an intensity could be represented by a bar graph or color state. In the case of the emoticon, this representation certainly does not need to represent a scientifically accurate emotion. The emoticon would simply represent a mathematical model or particular combination of the measured responses. For example a weighted combination of high heart rate and low galvanic skin responses would trigger the system to generate an emoticon representing passion.

In one embodiment in accordance with the invention, the entertainment content can be a video game with violent content and a user can be a teenager. Even though the entertainment content can be rated to be age appropriate for the user, it is more relevant to customize the flow and intensity of the game in line with the user's physiological response to the game. In this embodiment, when the system detects one of more among the user's pulse rate, heart rate or eye movements being outside of computed/determined threshold limits (or outside of limits for metrics which combine these parameters), then the algorithm or system recognizes that the user is in a hyperactive state and can change the game content to less violent or less demanding situations. For example, the game action could change from fight to flight of an action figure. Conversely, if the game action gets to be very boring as indicated by dropping heart rate, eye movement, etc., then the game can be made more exciting by increasing the pace or intensity of the action.

In another embodiment, the entertainment system can record the change in content flow and content nature in concordance with the user emotional response and can use such information to make decisions about how to structure the content when the user accesses the same content on a subsequent occasion. This form of customization or tailoring can make the content more appropriate for particular users. Different users can possibly use such a system for treatment, training or for mission critical situations. For example, fireman, police forces and military personnel can be chosen for critical missions based on their current emotional state in combination with a profile. In another example, emotional and mental patients can be tracked by psychologists based on emotions determined on a phone. With respect to healthcare and fitness, some people are more emotionally stable and able to handle rigorous work or training on some days as opposed to other days. Consider an example of a nuclear plant worker performing a critical task on a particular day. Management can use emotional state to choose the worker who is in the best emotional condition to perform the task.

Note, a profile as used in various embodiments herein can be a record of among all or portions of various inferred/estimated emotional states of the user (combination of one or more emotional states), time sequence of the emotional states and various stimulus contexts (such as a scene in a movie, a state of a video game, a type of music played, a difficulty level of a programming task, etc.) and the temporal relationship between the inferred state and the stimulus context. This profile can also include such external environmental information such as ambient conditions (lighting, loudness, humidity, weather, temperature, location, GPS input (a particular location the where person becomes excited), etc.). In addition, the profile can also include user identification information comprising of user id, age, gender, education, temperament, past history with the same or similar stimulus class etc.). The profile can then be saved in any of a variety of forms from simple object attribute data to more sophisticated probability density functions associated with neural networks or genetic algorithms. The complexity and sophistication of the storage method can be based on the device resource context and added value of the premium features.

In light of the foregoing description, it should be recognized that embodiments in accordance with the present invention can be realized in hardware, software, or a combination of hardware and software. A network or system according to the present invention can be realized in a centralized fashion in one computer system or processor, or in a distributed fashion where different elements are spread across several interconnected computer systems or processors (such as a microprocessor and a DSP). Any kind of computer system, or other apparatus adapted for carrying out the functions described herein, is suited. A typical combination of hardware and software could be a general purpose computer system with a computer program that, when being loaded and executed, controls the computer system such that it carries out the functions described herein.

In light of the foregoing description, it should also be recognized that embodiments in accordance with the present invention can be realized in numerous configurations contemplated to be within the scope and spirit of the claims. Additionally, the description above is intended by way of example only and is not intended to limit the present invention in any way, except as set forth in the following claims.

What is claimed is:

1. An electronic device, comprising:
a sensor for monitoring at least one current physiological measurement of a user;
a memory for storing a user profile containing information based on past physiological measurements of the user;
a presentation device for providing a presentation to the user; and
a processor coupled to the sensor and the presentation device, wherein the processor is programmed to alter the presentation based on the user profile and the at least one current physiological measurement of the user.

2. The electronic device of claim 1, wherein the user profile comprises at least one or more among a plurality of inferred or estimated emotional states of the user, a time sequence of emotional states, stimulus contexts, and a temporal relationship between the emotional state and the stimulus context.

3. The electronic device of claim 1, wherein the user profile further comprises recorded environmental conditions selected among the group comprising lighting, loudness, humidity, weather, temperature, and location.

4. The electronic device of claim 1, wherein the user profile comprises at least one among a user id, age, gender, education, temperament, and past history with the same or similar stimulus class.

5. The electronic device of claim 1, wherein the electronic device comprises at least one among a mobile phone, a smart phone, a PDA, a laptop computer, a desktop computer, an electronic gaming device, a gaming controller, a remote controller, a DVD player, an MP3 player, or a CD player.

6. The electronic device of claim 1, wherein the sensor for monitoring comprises at least one sensor for monitoring at least one among heart rate, pulse, blood oxygen levels, temperature, eye movements, body movements, breathing rates, audible vocalizations, skin conductivity, skin resistivity, Galvanic skin responses, audio level sensing, location, or force sensing.

7. The electronic device of claim 1, wherein the presentation device comprises at least one among a display, an audio speaker, a vibrator, or other sensory output device.

8. The electronic device of claim 1, wherein the electronic device further comprises a receiver and a transmitter coupled to the processor.
